(19) United States
(12) Patent Application Publication
(10) Pub. No.: US 2008/0177249 A1
(43) Pub. Date: Jul. 24, 2008

(54) CATHETER INTRODUCER SYSTEM

(76) Inventors: Richard R. Heuser, Phoenix, AZ (US); James D. Joye, Saratoga, CA (US)

Correspondence Address: KOLISCH HARTWELL, P.C., 520 SW YAMHILL STREET, Suite 200, PORTLAND, OR, 97204, US

(21) Appl. No.: 11/735,734

(22) Filed: Apr. 16, 2007

Related U.S. Application Data
(63) Continuation-in-part of application No. 11/656,898, filed on Jan. 22, 2007.
(60) Provisional application No. 60/887,277, filed on Jan. 30, 2007.

(51) Int. Cl. A61M 25/01

(52) U.S. Cl. 604/523

(57) ABSTRACT

Disclosed is an improved pointed instrument for insertion into blood vessels where an occlusion arises or terminates too closely to the insertion site for proper sheath placement. The pointed instrument exhibits supportive characteristics that allow for a sheath to be placed in an otherwise inaccessible insertion site. One embodiment of the present disclosure features a pointed instrument having a flexible portion, which is generally straight outside the body, but agglomerates once inside a blood vessel.

### CROSS REFERENCE TO RELATED APPLICATIONS

This application is a continuation-in-part of U.S. patent application Ser. No. 11/656,898, filed on Jan. 22, 2007, by Applicant Richard Heuser, the disclosure of which is incorporated by reference. This application also claims priority to Provisional Application Ser. No. 60/887,277, filed on Jan. 30, 2007, the disclosure of which is incorporated by reference.

### BACKGROUND

Catheters typically are inserted into a blood vessel through a sheath which is fixed in place relative to the blood vessel. The sheath protects the vessel and adjacent derma around the point of insertion while the catheter is advanced and withdrawn as necessary for the particular treatment being applied. The sheath is prepared for insertion by first placing a dilator within the lumen of the sheath and a needle or pointed wire within the lumen of the dilator. The needle or wire is used to pierce the derma and the blood vessel's wall and then is advanced into the blood vessel's lumen. Then the dilator is advanced along the wire without moving the needle or wire so that the tip of the dilator passes through the derma and blood vessel wall, expanding the diameter of the opening into the blood vessel.

At this point, with the dilator tip within the blood vessel lumen, the needle or wire may be withdrawn from the blood vessel. The sheath may then be advanced along the dilator, holding the dilator stationary, until the tip of the sheath is within the blood vessel's lumen. The sheath typically also expands the opening into the blood vessel. Once the sheath is in the blood vessel at a suitable location, the dilator may be withdrawn and the sheath braced in position, for example by taping the sheath to the patient's arm or leg adjacent the point of insertion. Then, the catheter is inserted through the sheath and into the blood vessel and maneuvered as necessary to provide the desired treatment.

The catheter's outer diameter (o.d.) in such a system is limited by the inner diameter of the sheath, which in turn is limited by the expansion provided by the dilator. A typical dilator-sheath introducer provides a dilator lumen allowing only a 3-French or 0.038 inch o.d. wire, and the dilator must be expanded outwardly relatively abruptly to reach even a modest outer diameter. The sheath lumen then is the same size as the outer diameter of the dilator, and typical size is only a 9-French or 0.118 inch, thus limiting the o.d. of the catheter to 9-French, which limits the types and capabilities of the catheters that can be used with such an introducer.

The dilator and sheath are typically formed of a plastic material and the tips of the dilator and sheath are sometimes damaged by insertion into the blood vessel. This may cause trauma to the derma and blood vessel, or require a second needle insertion if the tip is damaged to the extent it prevents suitable insertion.

The sheath typically includes an elastic collar adjacent a proximal end, through which the catheter is inserted. Such elastic collars are typically designed to allow the catheter to be advanced and withdrawn while providing some deterrence to blood leaking out through the sheath between the washer and the catheter. However, blood leakage past the washer is sometimes significant, causing unwanted complications to the catheterization procedure.

An issue also arises when a physician attempts to enter the vessel from the antegrade common femoral approach, or from the retrograde popliteal approach. An occlusion may arise or terminate in such close proximity to the access site that sheath or dilator placement is difficult.

### SUMMARY OF THE DISCLOSURE

A catheter introducer system is provided for inserting a sheath into a human blood vessel to provide a channel for a catheter to be moved within the blood vessel and to be held in a fixed position with minimum blood loss through the sheath. The catheter introducer system may include a trocar or other pointed instrument, such as a needle or pointed wire, for initially piercing the derma and the wall of the blood vessel. One or more dilators may surround the trocar and be sequentially inserted into the blood vessel after the trocar. The sheath surrounds the outermost of the dilators and is inserted last into the blood vessel. After removal of the trocar and dilator(s), the catheter may be inserted into the sheath and maneuvered within the blood vessel as necessary for the desired treatment.

When the catheter is at a desired location, it may be fixed longitudinally in place and blood loss through the sheath minimized by selective tightening of a hemostasis valve that includes a chuck that radially grips the catheter. The chuck may include a washer held between a base and a clamp and tightened therebetween, with beveled surfaces on the base, clamp, and/or the washer to provide for radial tightening of the chuck on the catheter.

The disclosure further describes an improved pointed instrument, such as a guidewire or trocar, for insertion into sites where an occlusion arises or terminates too closely to the insertion site for proper sheath or dilator placement. The pointed instrument exhibits supportive characteristics that allow for a sheath or dilator to be placed in an otherwise inaccessible insertion site. One embodiment of the present disclosure features a pointed instrument having a flexible portion, which is generally straight outside the body, but agglomerates once inside a blood vessel.

### BRIEF DESCRIPTION OF THE DRAWINGS

FIG. 1 is an isometric view of a catheter introducer system according to the present invention, including a dilator, a sheath, and a trocar.

FIG. 2 is an isometric view of the catheter introducer system as shown in FIG. 1, the system in this embodiment including two dilators.

FIG. 3 is an isometric view of the catheter introducer system as shown in FIGS. 1 and 2, the system in this embodiment including three dilators.

FIG. 4 is an isometric, exploded view of a proximal end of a sheath in accordance with the present invention, including a hemostasis valve with a chuck for variably gripping a catheter, the chuck including a base, a clamp coupled to the base by a threaded engagement, and a washer disposed between the base and the clamp.

FIG. 5 is a cross-sectional view of the proximal end of the sheath shown in FIG. 4, with the catheter inserted through the sheath and the washer therein, the clamp positioned to allow the catheter to move longitudinally through the sheath.

FIG. 6 is a cross-sectional view of the sheath as in FIG. 5, with the clamp positioned to tighten the washer onto the catheter.

FIG. 7 is a close-up pictorial view of the distal tip of the trocar of the catheter introducer system, showing an outer, hollow wire, an inner needle within the lumen of the wire and including a beveled tip extending out of the wire, and a boot at the tip of the wire.

FIG. 8 is a pictorial view of the trocar of FIG. 7 with the inner needle withdrawn into the wire and the boot closing to seal off the lumen of the wire.

FIG. 9 is a close-up end view of an alternative embodiment of the trocar of the catheter introducer system, showing a shutter at the tip of the wire that is open and a needle within the opening of the shutter.

FIG. 10 is a pictorial view of the trocar in the extended configuration shown in FIG. 9.

FIG. 11 is an end view of the trocar of FIG. 9 with the needle withdrawn into the wire and the shutter closed.

FIG. 12 is a pictorial view of the trocar in the withdrawn configuration of FIG. 11.

FIG. 13 is a side, cross-sectional view of the trocar of FIGS. 7-12 with the needle and wire tip inserted through the derma and into the blood vessel lumen of the patient, with the needle tip still extending out of the distal opening of the wire.

FIG. 14 is a side, cross-sectional view of the trocar as in FIG. 13 with the needle withdrawn from the wire and the wire returned to a flexible state within the blood vessel lumen.

FIG. 15 shows a pointed instrument according to another aspect of the present disclosure.

FIG. 16 depicts a pointed instrument of the present disclosure being inserted through a layer of skin into a blood vessel.

FIG. 17 depicts the pointed instrument of FIG. 16 further into the blood vessel, with the pointed instrument's flexible tip agglomerating within the blood vessel.

FIG. 18 shows the pointed instrument of FIGS. 16 and 17 inserted as far as it will go into the blood vessel.

FIG. 19 shows a sheath being inserted over the pointed instrument.

FIG. 20 shows a tubular introducer according to another aspect of the present disclosure approaching the skin and a blood vessel.

FIG. 21 shows the tubular introducer of FIG. 20 inserted through the derma and into the lumen of the blood vessel, with a pointed instrument extended through the tubular introducer and into the blood vessel.

FIG. 22 shows a dilator being advanced down the pointed instrument of FIG. 21.

### DETAILED DESCRIPTION OF THE INVENTION

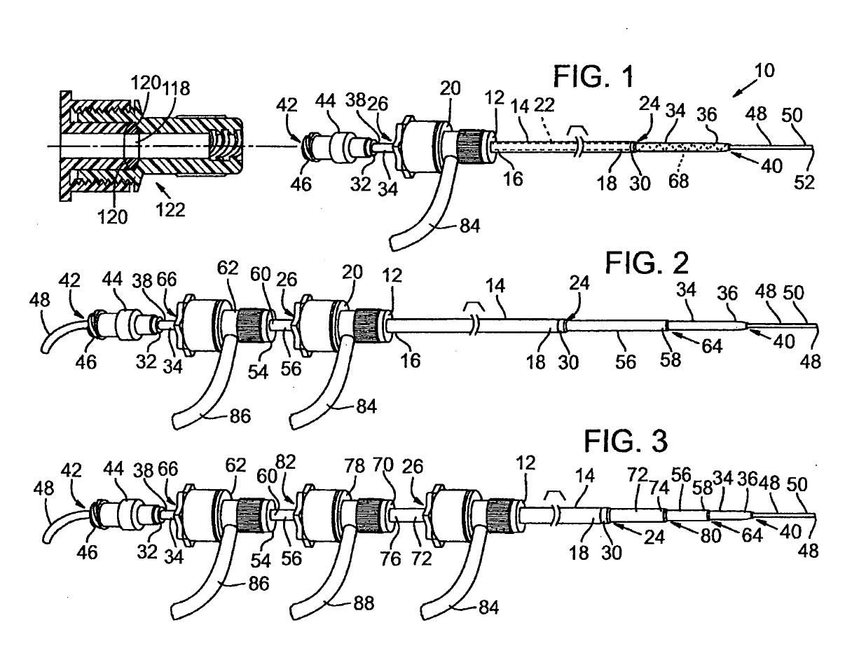

A catheter introducer system in accordance with the present invention is indicated generally at 10 in FIG. 1. Catheter introducer system 10 includes a sheath 12 having a generally elongate, typically cylindrical body 14 with a proximal end 16 and a distal end 18. Body 14 of sheath 12 is coupled at proximal end 16 to a housing 20. A central, typically cylindrical lumen 22 is defined within sheath 12 extending out through a distal opening 24 and a proximal opening 26. Sheath 12 is thus configured to receive a catheter 28 (FIG. 5) for longitudinal movement through and torsional movement within lumen 22.

Body 14 of sheath 12, is typically composed substantially of a flexible material, such as a plastic, polymeric material, preferably a hydrophilic material. Body 14 may include a reinforcement, such as stainless steel ring 30, to prevent buckling or crimping of distal end 18 at opening 24 when sheath 12 is inserted into a human blood vessel. The stainless steel ring 30 on the outermost sheath is radiopaque, as would be understood by those of ordinary skill in the art.

Catheter introducer system 10 further includes a first dilator 32 configured to be inserted into and disposed substantially within lumen 22 of sheath 12. First dilator 32 includes a generally elongate, typically cylindrical body 34 having a distal end 36 and a proximal end 38. Dilator 32 typically also includes a lumen interconnecting a distal opening 40 and a proximal opening 42, which typically is located in a housing 44 coupled to body 34 adjacent proximal end 38. Housing 44 may be provided with a luer lock fitting 46, which typically includes a double threaded engagement portion for coupling to mating devices.

Sheath 12 and dilator 32 typically are generally cylindrical, as noted above, typically with tapering adjacent distal ends 18, 36 to provide a smooth transition as the ends enter the blood vessel. Alternatively sheath 12 and dilator 32, and one or more intermediate dilators (to be described below), may be tapered substantially along the entire length of their bodies, preferably providing a more gradual, or additional, tapering as compared to the tapered distal ends. In either case, the sheath and dilators are preferably provided with a smooth transition between the body of one dilator and the distal end of the next outer sheath or dilator to prevent the distal end from snagging on the derma or blood vessel during insertion into the blood vessel.

Dilator 32 is preferably longer than sheath 12 so that distal end 36 and proximal end 38 of body 34 of dilator 32 extend out of the distal and proximal openings of sheath 12, as shown in FIG. 1. As an example, dilator 32 may be on the order of about 24 cm long and sheath 12 may be in the range of about 16 cm to about 20 cm, with other lengths selectable as appropriate for a particular application.

Catheter introducer system 10 may also include a trocar 48 inserted through dilator 32 and including a distal end 50 with a sharp, pointed tip 52. Trocar 48 is inserted through dilator 32 to create the initial entrance through the derma and into the blood vessel, and trocar 38 then is withdrawn after distal end 36 of dilator 32 is advanced into the blood vessel.

As shown in FIG. 2, catheter introducer system 10 may include a second, intermediate dilator 54 between the first dilator 32 and sheath 12. Second dilator 54 typically includes a generally elongate, substantially cylindrical body 56 with a distal end 58 and a proximal end 60. A housing 62 is coupled to body 56 adjacent proximal end 60. A central lumen, similar to that for sheath 12 and first dilator 32 interconnects a distal opening 64 and a proximal opening 66.

Second dilator 54 preferably has a length greater than that of sheath 12 and less than that of first dilator 32. Thus, if first dilator 32 is about 24 cm, second dilator 54 is about 20 cm and sheath 12 is about 16 cm in length. The lengths of all the components of the catheter introducer system may be adapted for the particular application.

Preferably the dilator(s) and sheath are formed of less stiff, more flexible material as one progresses from innermost to outermost. E.g., first dilator 32 may be formed with the most stiff material, and may include a reinforcement, such as a wire 68 (FIG. 1), embedded or otherwise affixed to the material in a coil, web, or other suitable pattern to provide a stiffener. Second dilator 54 is typically formed of a less stiff material, while sheath 12 is formed of a still softer material so that the sheath/dilator bodies tend to be less traumatic to the blood vessel as they get larger. Second dilator, and other intermediate dilators, and sheath are typically progressively more hydrophilic as one proceeds outwardly. Ring 30, described above, helps to insure that the soft material of sheath 12 will not buckle or flare if the sheath tip catches or snags on the derma on insertion into the blood vessel. A ring may also be provided on the tips of one or more of the dilators in addition to or instead of on sheath 12.

With a two-dilator/one-sheath system, some typically available inner diameters (i.d.) in French sizes are shown in the following table:

| Two-dilator/ | | | |
|---|---|---|---|
| one-sheath | First dilator i.d. | Second dilator i.d. | Sheath i.d. |
| Ex. 1 | 3 | 4 | 5 |
| Ex. 2 | 3 | 5 | 6 |
| Ex. 3 | 3 | 5 | 7 |
| Ex. 4 | 3 | 5 | 8 |
| Ex. 5 | 3 | 5 | 9 |
| Ex. 6 | 3 | 8 | 16 |

Other sizes may be used as appropriate for a particular application.

As shown in FIG. 3, catheter introducer system 10 may include a third, intermediate dilator 70 between the second dilator 54 and sheath 12. Second dilator 70 typically includes a generally elongate, substantially cylindrical body 72 with a distal end 74 and a proximal end 76. A housing 78 is coupled to body 72 adjacent proximal end 76. A central lumen, similar to that for the sheath and the first and second dilators interconnects a distal opening 80 and a proximal opening 82.

Third dilator 70 is preferably intermediate in length, in softness, and in hydrophilicity between second dilator 54 and sheath 12. With a three-dilator/one-sheath system, some typically available inner diameters (i.d.) in French sizes are shown in the following table:

| Three-dilator/ | First | Second | | |
|---|---|---|---|---|
| one-sheath | dilator i.d. | dilator i.d. | Third dilator i.d. | Sheath i.d. |
| Ex. 7 | 3 | 5 | 7 | 10 |
| Ex. 8 | 3 | 5 | 9 | 11 |
| Ex. 9 | 3 | 5 | 9 | 12 |
| Ex. 10 | 3 | 5 | 9 | 13 |
| Ex. 11 | 3 | 5 | 9 | 14 |
| Ex. 12 | 3 | 5 | 9 | 15 |
| Ex. 13 | 3 | 5 | 9 | 16 |
| Ex. 14 | 3 | 5 | 9 | 17 |
| Ex. 15 | 3 | 5 | 9 | 18 |

Other sizes may be used as appropriate for a particular application.

It will be seen from the foregoing that the system may include any number of dilators to reach a desired sheath size, and, for example, a four-dilator/one-sheath system could use the following sizes:

| Four-dilator/ | First dilator | 2nd dilator | 3rd dilator | 4th dilator | Sheath |
|---|---|---|---|---|---|
| one-sheath | i.d. | i.d. | i.d. | i.d. | i.d. |
| Ex. 16 | 3 | 5 | 9 | 12 | 19 |
| Ex. 17 | 3 | 5 | 9 | 12 | 20 |
| Ex. 18 | 3 | 5 | 9 | 12 | 21 |
| Ex. 19 | 3 | 5 | 9 | 12 | 22 |
| Ex. 20 | 3 | 5 | 9 | 12 | 23 |
| Ex. 21 | 3 | 5 | 9 | 15 | 24 |
| Ex. 22 | 3 | 5 | 9 | 15 | 26 |
| Ex. 23 | 3 | 5 | 9 | 15 | 28 |

Other sizes may be used as appropriate for a particular application.

Sheath 12 as shown in FIGS. 1-3 includes a side branch or arm 84 in housing 20, which is commonly used for the introduction of intravenous fluid or medicine during a catheterization procedure. As shown in FIGS. 2 and 3, second dilator 54 includes a side branch 86 in housing 62 and third dilator 70 includes a side branch 88 in housing 78. Although the term dilator is commonly used to refer to a device without a side branch, FIGS. 2 and 3 illustrate that dilator, as used herein, can refer to a device either with or without a side branch, and the catheter introducer system may be used with sheaths and dilators including any combination of side branches or lack thereof.

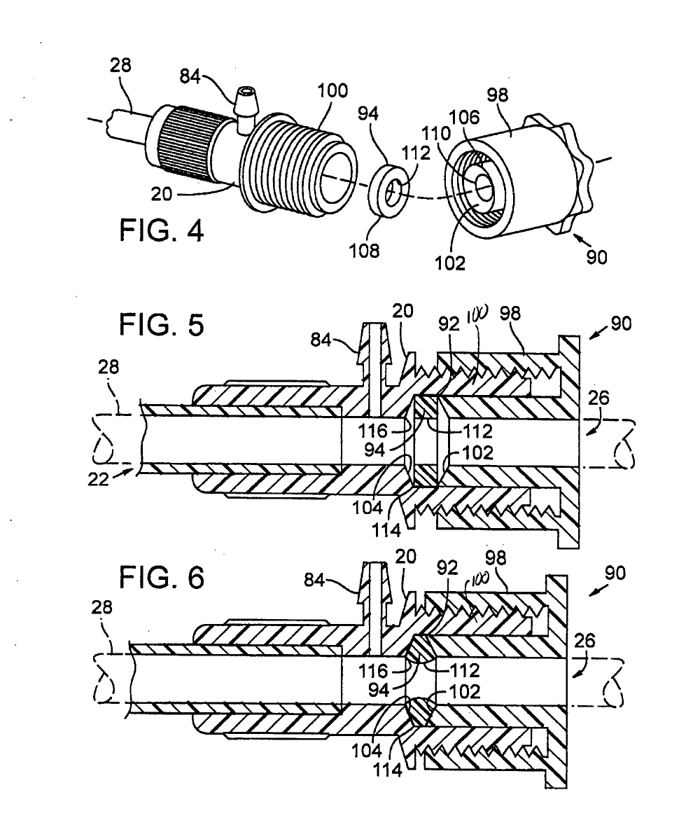

As best seen in FIGS. 4-6, housing 20, which may be identical in the following respects to the housings on any or all of the dilators, includes a hemostasis valve, indicated generally at 90, for preventing blood leaks between catheter 28 and housing 20. Such blood leaks typically flow along inner lumen 22 and out proximal opening 26. Housing 20 is shown in FIGS. 4-6 with all of the smaller dilators and the trocar withdrawn, and, in FIGS. 5 and 6 with catheter 28 inserted.

Hemostasis valve 90 includes a chuck 92 for selectively gripping radially onto catheter 28. Chuck 92 includes a collar, such as flexible, elastomer washer 94, defining an opening 96 configured to receive catheter 28 therethrough. Preferably washer 94 fits snugly around catheter 28 in a first configuration, shown in FIG. 5, allowing catheter 28 to be moved longitudinally therethrough.

Valve 90 thus provides for a friction fit around the catheter. The friction fit is selectively adjustable to vary between a first configuration for longitudinal movement of the catheter through the valve and a second configuration for holding the catheter longitudinally in place. A physician operating the catheter introducer system may selectively switch valve 90 to the second configuration, typically when catheter 28 is in a desired location within the blood vessel to perform a catheter procedure, such as installing a stent or applying RF energy at the distal tip of the catheter.

Preferably the physician selects the second configuration by grasping a threaded cap 98 of valve 90, and rotating to tighten cap 98 onto a threaded portion 100 of housing 20. Such tightening of cap 98 drives a clamp 102 within cap 98 to squeeze washer 94 between clamp 102 and a base 104 in housing 20. Preferably, clamp 102 is a sloped annular ring providing a beveled surface within cap 98. The ring preferably has an outer circumference 106 generally coincident with an outer circumference of washer 92, and an inner circumference 110 generally coincident with an inner circumference 112 of washer 92. Preferably, base 104 is a sloped annular ring, providing a beveled surface that includes an outer circumference 114 and an inner circumference 116 coincident with corresponding circumferences of the washer and the clamp. Inner circumference 112 of washer 92 is nominally at least as large as the o.d. of catheter 28, although it can be smaller to provide a tighter nominal friction fit.

In the second configuration, shown in FIG. 6, cap 98 has been tightened onto housing 20 so that clamp 102 and base 104 are closer together than a nominal width of washer 92. Thus washer 92 is pressed radially inwardly by the sloping faces of clamp 102 and base 104, which tends to reduce the washer's inner circumference 112 and to close opening 96, thereby tightening washer 92 onto catheter 28. Valve 90 may be selectively adjusted by the physician to any degree of tightness, thereby allowing the physician to select an optimal combination of leakage prevention and ease of catheter longitudinal and torsional movement. Preferably, with cap 98 fully tightened, substantially no blood leaks between catheter 28 and washer 92 and catheter 28 is substantially locked longitudinally in place.

Washer 92 is shown in FIGS. 4-6 as a flat washer. Washer 92 may alternatively have one or two sloped or beveled surfaces which may correspond to the sloped faces of clamp 102 and base 104. A washer 118 with two sloped faces 120 is shown in FIG. 1 incorporated into a hemostasis valve, indicated generally at 122, on first dilator 32. Valve 122 operates in a manner generally similar to that of valve 90.

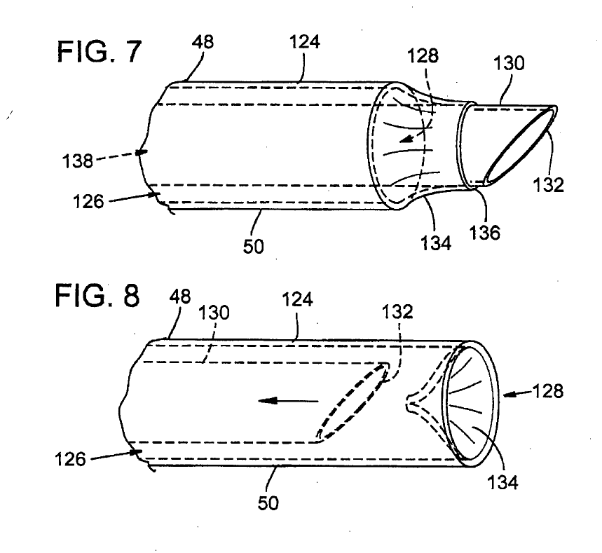

FIGS. 7-14 show embodiments of trocar 48, in particular the structure at distal end 50 of trocar 48. Trocar 48 may include a hollow, preferably flexible wire 124 defining a central lumen 126 extending out to a distal opening 128. A hollow, preferably stiff needle 130 provided with a sharp tip, such as beveled end 132, may be inserted in lumen 126. In an operative condition, as shown in FIG. 7, tip 132 will extend out of opening 128 of wire 124. A sealing mechanism, such as flexible cuff or boot 134 may be coupled to wire 124 at opening 128. Sealing mechanism 134 includes an opening 136 which stretches to allow needle tip 132 to extend through the sealing mechanism in the operative condition. Needle 130 includes a proximal end extending out of the proximal end of trocar 48, which extends out of proximal opening 42 of dilator 32 (see FIGS. 1-3). Needle 130 also includes a central lumen 138 extending from tip 132 to the proximal end.

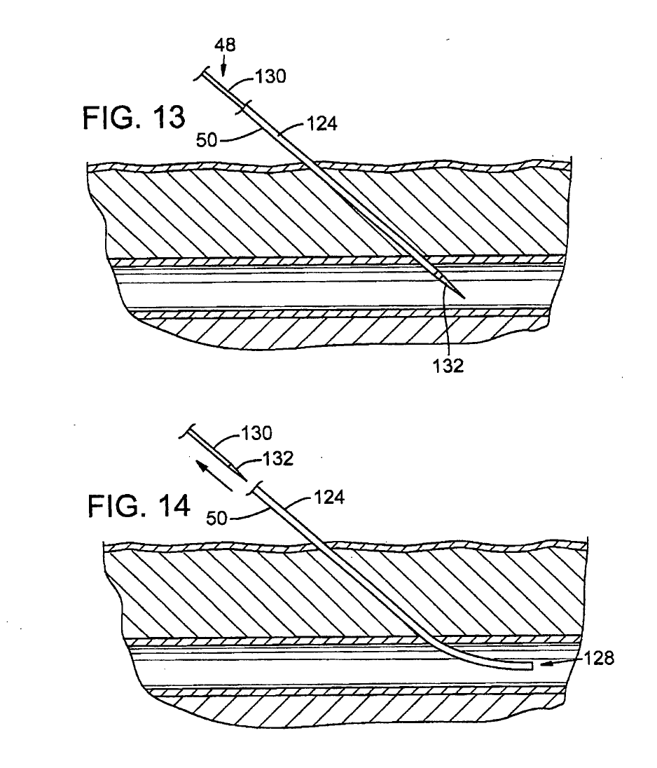

As shown in FIGS. 13 and 14, the physician may use trocar 48 with needle tip 132 extending out of wire 124 to insert the trocar tip through the patient's derma and into the blood vessel. FIG. 13 shows that the flexible wire with the stiff needle inserted therethrough is stiff and insertable into the blood vessel along with the needle tip. The physician can verify that the needle tip is in the blood vessel by the blood that will flow into lumen 138 of needle 130 and out the proximal end.

Once the wire and needle are verified as inserted into the lumen of the blood vessel, the physician can pull on the proximal end of the stiff needle, withdrawing it from the trocar tip, as shown in FIG. 14, at which time wire 124 will again become flexible. The flexible wire presents a less dangerous and traumatic device within the blood vessel, as compared to a stiff wire or needle. The withdrawn needle is also shown in FIG. 8, where it can be seen that sealing mechanism 134 has closed to prevent further blood flow through trocar 48.

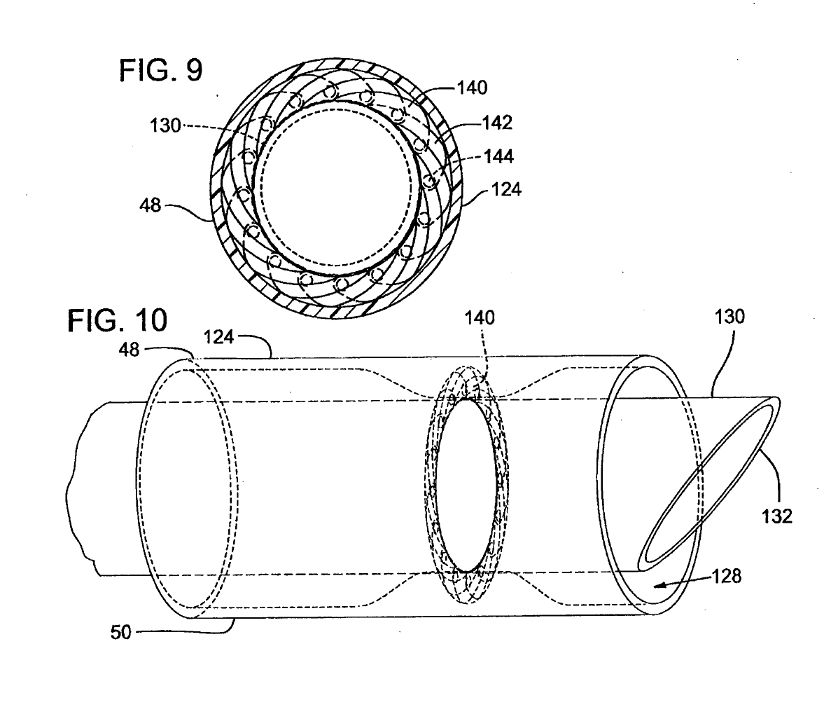

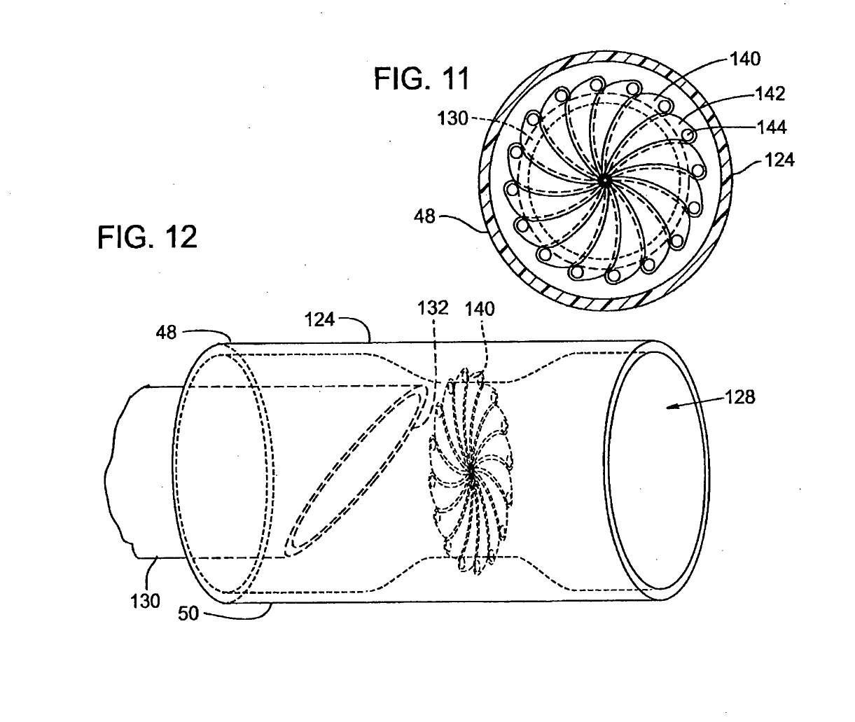

Another embodiment of a sealing mechanism is shown in FIGS. 9-12, where wire 124 includes adjacent opening 128 a shutter 140. A plurality of shutter vanes 142 may be mounted on hinges 144 and preferably may be nominally biased to a closed position (FIGS. 11 and 12). Shutter 140 can be opened by inserting tip 132 of needle 130 through the shutter, as is shown in FIGS. 9 and 10. The physician uses the embodiment of FIGS. 9-12 in the same manner as that of FIGS. 7 and 8, inserting the needle tip through the derma and into the lumen of the blood vessel and then withdrawing the needle, allowing the shutter to close and the wire 124 to go limp, as shown in FIGS. 13 and 14.

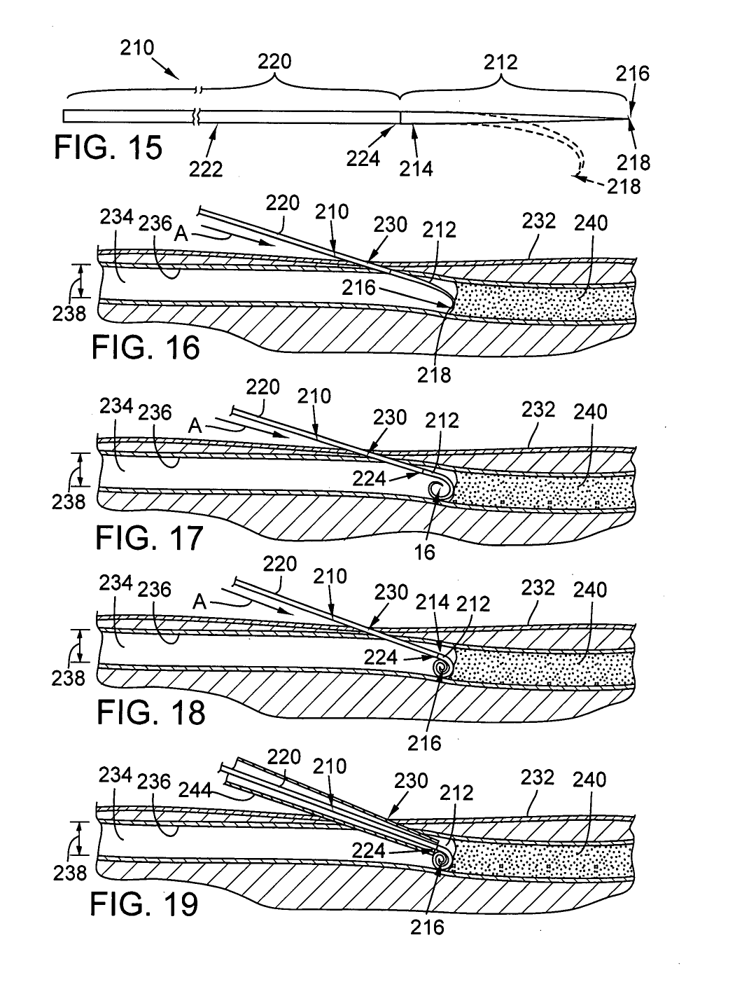

Another aspect of the present disclosure is shown in FIG. 15, where a pointed instrument 210 is shown having a flexible portion 212 with a first proximal end 214 and a first distal end 216. The distal end 216 may have a sharp tip 218. The pointed instrument 210 also includes an inflexible portion 220 having a second proximal end 222 and a second distal end 224, the second distal end 224 being adjacent to the first proximal end 214.

FIG. 16 depicts the pointed instrument 210 of FIG. 15 being inserted into a blood vessel 234 formed by vessel wall 236 and having a diameter 238. An entry point 230 is a position on the derma 232 that is within close proximity to the vessel 234. The vessel 234 is blocked in close proximity to the entry point 230 by an occlusion 240.

The pointed instrument 210 has been inserted through the derma 232 and the vessel wall 236 to a position within the blood vessel 234. The first distal end 216 is seen contacting the occlusion 240. Due to its inherent flexibility, the flexible portion 212 begins to agglomerate (i.e. bunch up), instead of piercing the occlusion 240 or the lower portion of the vessel wall 236.

In one embodiment the flexibility may stem from the flexible portion 212 being at least partially comprised of hydrophilic materials. Constructing the flexible portion 212 at least partially of hydrophilic materials would cause it to become malleable upon insertion into fluid. For instance, insertion into the blood vessel 234, which may be filled with blood, would cause this embodiment of the flexible portion 212 to become malleable. Pressing the distal end 216 of the malleable flexible portion 212 against any foreign surface (i.e. the occlusion 240) would cause the portion 212 to agglomerate upon itself.

Alternatively, the flexible portion 212 may be comprised of a temperature sensitive nitinol alloy. The flexible first portion 212 may have a thermal characteristic, whereby it may be configured to be inflexible when the thermal characteristic is below a first temperature and to agglomerate when the thermal characteristic is above a second temperature.

In one instance, the flexible first portion 212 may be configured to be inflexible when the thermal characteristic is within a temperature range typically found in an operating room. The flexible portion 212 may likewise be configured to be flexible when the thermal characteristic is at a temperature typically found in a human body.

In another instance, the flexible first portion 212 may be configured to be inflexible when its thermal characteristic is approximately room temperature. Room temperature may vary from a temperature as low as 16° Celsius to a temperature as high as 28° Celsius.

FIG. 17 depicts the pointed instrument 210 being further inserted into the vessel 234, so that the second distal end 224 of the inflexible portion 220 is closer to the occlusion 240 than it was in FIG. 16.

FIG. 18 shows the pointed instrument 210 being inserted as far as it will go into the vessel 234. The second distal end 224 of the inflexible portion 220 is seen in close proximity with the occlusion 240, thus preventing the pointed instrument 210 from being advanced further into the vessel 234.

FIG. 19 depicts a sheath 244 inserted over the pointed instrument 210, with the distal tip of the sheath 244 abutting or being in close proximity with the occlusion 240 and the second distal end 224 of the inflexible portion 220.

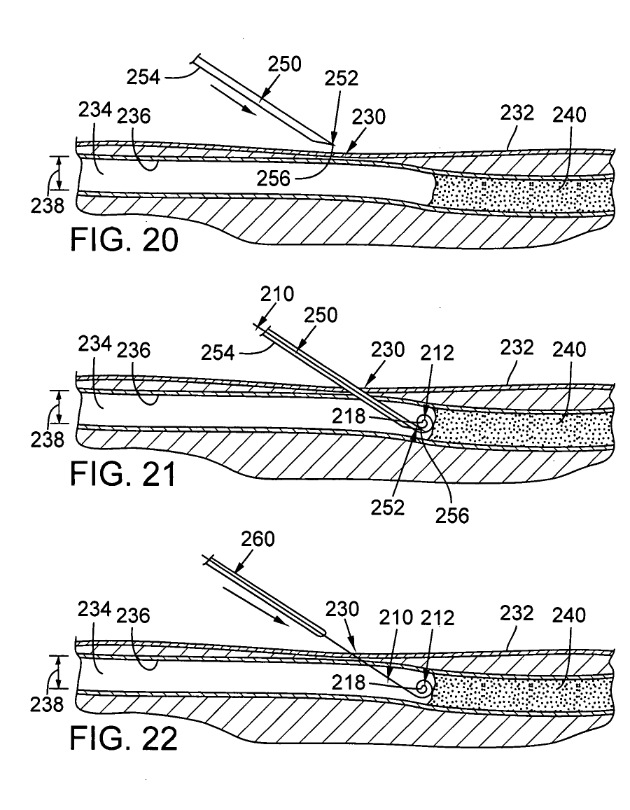

FIGS. 20-22 depict another embodiment, where the flexible first portion 212 is biased towards agglomeration or a coiled configuration. In such an embodiment, a tubular introducer 250 having a distal end 252, a proximal end 254, and a sharp tip 256 may be used to pierce the derma 232 and the vessel wall 236, so that the distal end 252 of the introducer 250 may be extended into the lumen of the blood vessel 234. One example of a suitable tubular introducer 250 is similar to the hollow needle 130 of FIGS. 7-14. Another example can be seen in FIGS. 20-21.

Once the distal tip 252 of the introducer 250 is extended into the lumen of the vessel 234, the pointed instrument 210 may be extended from within the introducer 250, and being biased to an agglomerated configuration, the flexible portion 212 will agglomerate (as seen in FIG. 21). The physician may continue to extend the pointed instrument 210 until the second distal end 224 of the inflexible portion 220 is at a position adjacent to the agglomerated flexible portion 212.

FIG. 22 shows a possible next step, wherein the tubular introducer 250 is removed, and a dilator 260 is advanced down the pointed instrument 210 into the blood vessel 234.

It is believed that the following claims particularly point out certain combinations and subcombinations that are directed to one of the disclosed disclosures and are novel and non-obvious. Disclosures embodied in other combinations and subcombinations of features, functions, elements and/or properties may be claimed through amendment of the present claims or presentation of new claims in this or a related application. Such amended or new claims, whether they are directed to a different disclosure or directed to the same disclosure, whether different, broader, narrower or equal in scope to the original claims, are also included within the subject matter of the disclosures of the present disclosure.

1. A pointed instrument for use in a micropuncture kit, the pointed instrument comprising:
a flexible portion having a first distal end insertable into a position within a blood vessel adjacent to an occlusion and a first proximal end, the flexible portion having an inflexible elongate shape outside the body, and being configured to agglomerate upon insertion into a blood vessel; and
an inflexible portion having a second proximal end and a second distal end adjacent to the first proximal end of the flexible portion, the inflexible portion insertable to a position in the blood vessel wherein the second distal end is adjacent to the agglomerated flexible portion.

2. The pointed instrument of claim 1, wherein the flexible portion is hydrophilic.

3. The pointed instrument of claim 1, wherein the flexible portion is comprised of nitinol.

4. The pointed instrument of claim 3, wherein the flexible portion includes a thermal characteristic wherein the flexible portion is configured to be inflexible when the thermal characteristic is below a temperature, and the flexible portion is configured to be flexible when the thermal characteristic is above a second temperature.

5. The pointed instrument of claim 4, wherein the first temperature less than or equal to 28 degrees Celsius, and the second temperature is greater than or equal to 35 degrees Celsius.

6. The pointed instrument of claim 4, wherein the flexible portion is configured to be inflexible when the thermal characteristic is at room temperature and to agglomerate when the thermal characteristic is at body temperature.

7. The pointed instrument of claim 1, further comprising a tubular introducer which maintains the flexible portion in a generally elongate shape until the flexible portion exits the introducer and enters the blood vessel, the flexible portion being biased towards an agglomerated configuration.

8. An introducer and guidewire assembly for creating access into a blood vessel, the assembly comprising:
a tubular introducer having a lumen and a sharp tip, the introducer being insertable through a layer of skin into the lumen of the blood vessel; and
a guidewire disposed within and extendable from the lumen of the introducer, the guidewire having a flexible portion adjacent to its distal end which is nominally biased towards an agglomerated configuration, and an inflexible portion adjacent to its proximal end, the guidewire being insertable into a position adjacent an occlusion within the blood vessel wherein the flexible portion is agglomerated and the inflexible portion is at least partially extended into the vessel.

9. A catheter introducer system comprising:
a pointed instrument having a proximal end, a distal end, and a flexible portion adjacent to its distal end which is nominally biased towards an agglomerated configuration;
a first dilator defining a generally elongate body having a sharp distal end and a proximal end, wherein the pointed instrument is disposed substantially within the lumen of the first dilator with the distal end of the pointed instrument extendable through a distal opening of the first dilator;
a second dilator defining a generally elongate body having a distal end and a proximal end, an opening adjacent each end, and an open lumen communicating with the openings, wherein the first dilator is disposed substantially within the lumen of the second dilator with the distal end of the first dilator extending through the distal opening of the second dilator; and
a sheath defining a generally elongate body having a distal end and a proximal end, an opening adjacent each end, and an open lumen communicating with the openings, wherein the second dilator is disposed substantially within the lumen of the sheath with the distal end of the second dilator extending through the distal opening of the sheath.

10. A catheter introducer system comprising:
a pointed instrument including:
a flexible portion having a first distal end insertable into a position within a blood vessel adjacent to an occlusion and a first proximal end, the flexible portion having an inflexible elongate shape outside the body, and being configured to agglomerate upon insertion into a blood vessel; and
an inflexible portion having a second proximal end and a second distal end, the second distal end being adjacent to the first proximal end of the flexible portion, and the inflexible portion being insertable to a position in the blood vessel wherein the second distal end is adjacent to the agglomerated first flexible portion; and
a first dilator defining a generally elongate body having a sharp distal end and a proximal end, wherein the pointed instrument is disposed substantially within the lumen of the first dilator with the distal end of the pointed instrument extendable through a distal opening of the first dilator;
a second dilator defining a generally elongate body having a distal end and a proximal end, an opening adjacent each end, and an open lumen communicating with the openings, wherein the first dilator is disposed substantially within the lumen of the second dilator with the distal end of the first dilator extending through the distal opening of the second dilator; and
a sheath defining a generally elongate body having a distal end and a proximal end, an opening adjacent each end, and an open lumen communicating with the openings, wherein the second dilator is disposed substantially within the lumen of the sheath with the distal end of the second dilator extending through the distal opening of the sheath.
